(19) United States
(12) Patent Application Publication
(10) Pub. No.: US 2009/0049856 A1
(43) Pub. Date: Feb. 26, 2009

(54) Working fluid of a blend of 1,1,1,3,3-pentafluoropane, 1,1,1,2,3,3-hexafluoropropane, and 1,1,1,2-tetrafluoroethane and method and apparatus for using

(75) Inventor: Andrew Sun, Suzhou (CN)

Correspondence Address: Honeywell International Inc.;Patent Services Department, 101 Columbia Road, Morristown, NJ, 07962, US

(73) Assignee: Honeywell International Inc.

(21) Appl. No.: 11/894,134

(22) Filed: Aug. 20, 2007

(51) Int. Cl. F25B 13/00; C07C 19/08; C09K 5/04; F25B 1/08

(52) U.S. Cl. 62/324.1; 252/67; 570/134; 62/498; 62/502

(57) ABSTRACT

There is a working fluid for a heating and cooling. The fluid is a blend of about 1% to about 98% by mass 1,1,1,3,3-pentafluoropane, about 1% to about 98% by mass 1,1,1,2,3,3-hexafluoropropane, and about 1% to about 98% by mass 1,1,1,2-tetrafluoroethane. The 1,1,1,3,3-pentafluoropane, 1,1,1,2,3,3-hexafluoropropane, and 1,1,1,2-tetrafluoroethane are about 90% or more by mass of the blend. There are also an apparatus that uses the blend and methods for heating and cooling using the blend.

### BACKGROUND OF THE INVENTION

1. Field of the Invention

The present invention relates to a working fluid for use in a heating/cooling apparatus. The present invention further relates to a working fluid of a blend of 1,1,1,3,3-pentafluoropane (R245fa), 1,1,1,2,3,3-hexafluoropropane (R236ea), and 1,1,1,2-tetrafluoroethane (R134a). The present invention still further relates to methods for heating or cooling using the working fluid. The present invention still yet further relates to an apparatus for heating or cooling having the working fluid.

2. Description of the Related Art

Heat pumps have been used to upgrade low-grade thermal energy, such as that derived from air, soil, surface water or underground water, geothermal energy, solar energy, and industrial exhaust heat and process streams, to high-grade thermal energy via a thermodynamic cycle. A heat pump system has a compressor that imparts energy to the low-grade thermal stream. Heat pump systems use a working fluid, i.e., a refrigerant, to facilitate the generation and transfer of heat over the thermodynamic cycle. Heat pump systems have been used for both heating and cooling purposes.

Historically, chlorofluorocarbons such as trichlorofluoromethane (CFC-11), 1,1,2-trichlorotrifluoroethane (CFC-113) and 1,2-dichloro-1,1,2,2-tetrafluoroethane (CFC-114) were used as working fluids in heat pumps, refrigerators, and other heating/cooling devices and machines. Due to elevated levels of Ozone Depletion Potential (ODP) and Global Warming Potential (GMP) the foregoing working fluids exhibit, their use has largely ended.

Chlorofluorocarbons have been replaced in heating and cooling applications by other working fluids that exhibit lower ODP and GMP, such as hydrochlorofluorocarbons and hydrofluorocarbons. Such working fluids include chlorodifluoromethane (R-22), R-407C, R-410A, and 1,1,1,2-tetrafluoroethane (R-134a). R-407C is a blend of difluoromethane (R-32), 2-chloro-1,1,1,2-tetrafluoroethane (R-124), R-134a, 1-Chloro-1,1-difluoroethane (R142b). R-410A is a blend of R-22 and pentafluoroethane (R-125).

The replacement working fluids do not provide the same operating range in middle to high heating temperatures that chlorofluorocarbon working fluids do. Of particular interest are middle-high temperatures, i.e., condensing temperatures from 70° C. to 100° C. and high temperatures, i.e., condensing temperatures greater than 100° C. For instance, for R22, R407c and R410A, the highest condensing temperature is 65° C. For R134a, the highest condensing temperature attainable is 73° C. When condensing temperatures exceed the limit, cycle performance deteriorates and risk of accidents increase due to excessive discharge pressures and temperatures (from the compressor).

It would be desirable to have a working fluid that exhibits low ODP and GWP and provides excellent thermal performance in the middle and high temperature ranges, particularly in middle-high condensation temperature range of 70° C. to 100° C. It would be further desirable to have a working fluid that is useful in heat pump systems and other heating/cooling machines such as air-conditioning systems and chillers.

### SUMMARY OF THE INVENTION

According to the present invention, there is provided a working fluid for heating and cooling. The fluid is a blend of about 1% to about 98% by mass 1,1,1,3,3-pentafluoropane (R245fa), about 1% to about 98% by mass 1,1,1,2,3,3-hexafluoropropane (R236ea), and about 1% to about 98% by mass 1,1,1,2-tetrafluoroethane (R134a). The 1,1,1,3,3-pentafluoropane, 1,1,1,2,3,3-hexafluoropropane, and 1,1,1,2-tetrafluoroethane are about 90% or more by mass of the blend.

Further according to the present invention, there is provided a heating/cooling apparatus. The apparatus has a compressor, a condenser, an expansion element, and an evaporator in series in a cycle. The apparatus further has therein a working fluid of a blend of about 1% to about 98% by mass R245fa, about 1% to about 98% by mass R236ea, and about 1% to about 98% by mass R134a. The R245fa, R236ea, and R134a are about 90% or more by mass of the blend.

Further according to the present invention, there is provided a method for heating/cooling. The method has the steps of (a) evaporating a working fluid in the form of a lower pressure liquid to form a lower pressure vapor, (b) compressing the lower pressure vapor to a higher pressure vapor, (c) condensing the higher pressure vapor to a higher pressure liquid, (d) expanding the higher pressure liquid to a lower pressure liquid; and (e) recycling the lower pressure liquid to step a). The working fluid includes a blend of about 1% to about 98% by mass R245fa, about 1% to about 98% by mass R236ea, and about 1% to about 98% by mass R134a. The R245fa, R236ea, and R134a are about 90% or more by mass of the blend.

Further according to the present invention, there is provided a method of cooling. The method includes the step of evaporating a blend of about 1% to about 98% by mass R245fa, about 1% to about 98% by mass R236ea, and about 1% to about 98% by mass R134a. The R245fa, R236ea, and R134a are about 90% or more by mass of the blend.

Further according to the present invention, there is provided a method of heating. The method includes the step of condensing a blend of about 1% to about 98% by mass R245fa, about 1% to about 98% by mass 1,1,1,2,3,3-hexafluoropropane, and about 1% to about 98% by mass 1,1,1,2-tetrafluoroethane. The R245fa, R236ea, and R134a are about 90% or more by mass of the blend.

### DETAILED DESCRIPTION OF THE INVENTION

The blend of the present invention has three components, R245fa, R236ea, and R134a. The blend has about 1 to about 98%, preferably about 2 to about 40%, and most preferably about 10% to about 40% by mass R245fa. The blend also has about 1 to about 98%, preferably about 2% to about 40%, and most preferably about 10% to about 40% by mass of R236ea. The blend also has about 1 to about 98%, preferably about 10% to about 85%, and most preferably about 20% to about 70% by mass of R134a. R245fa, R236ea, and R134a are about 90% or more and preferably about 95% or more by mass of the blend.

The blend of the present invention may also have minor amounts, i.e., up to about 10% and preferably up to about 5% by mass of refrigerant components other than R245fa, R236ea, and R134a that exhibit low ODP and GWP. Such components will typically be hydrochlorofluorocarbons and hydrofluorocarbons. Suitable components include, but are not limited to, R-22, R-32, 1,1-dichloro-2,2,2-trifluoroethane (R123), R-124, R-125, R142b, 1,1-difluoroethane (R152a), 1,1,2-trifluoroethane (R143), 1,1,1-trifluoroethane (R143a), 1,1,2,2,3-pentafluoropropane (R245ca), R-407C, and R-410A.

Another aspect of the present invention is an apparatus and method for heating or cooling employing the blend of R245fa, R236ea, and R134a. The apparatus operates via a vapor compression cycle, which comprises four basic processes: evaporation, compression, condensation and expansion. The apparatus has the following mechanical units: a compressor, a condenser, an expansion element, and an evaporator. The evaporator and condenser are heat exchangers in function. In some instances, the apparatus may have additional optional mechanical units, such as a subcooler, an oil separator, and an accumulator.

Evaporation takes place in the evaporator. In evaporation, heat is absorbed by the blend in the evaporator, i.e., cooling capacity is outputted. In the instance of a heat pump, the heat source for the evaporator may be low-grade thermal energy that is to be converted to high-grade thermal energy. In the instance of a refrigerator or a chiller, the evaporator absorbs heat from its environment and functions as a cooling source. The blend enters the evaporator as a low pressure liquid, absorbs heat at dew point temperature to become a superheated vapor. The dew point temperature is lower than the temperature of the heat source outside the evaporator. A positive evaporating pressure is maintained in the evaporator relative to the outside thereof to prevent air or moisture from entering or infiltrating.

After leaving the evaporator, the low-pressure superheated vapor enters the compressor through a suction line and is compressed to a high pressure. The compressor effects compression through consumption of electrical power or a mechanical energy source, such as a combustion engine. If desired or necessary, vapor temperature at the discharge of the compressor can be controlled via injection of liquid blend into the suction line, regulation of pressure ratio of output to input, or regulation of discharge pressure.

Condensation takes place in the condenser. In condensation, heat is released or devolved by the blend in the condenser, i.e., heat is outputted in the form of high-grade thermal energy (or at least higher grade, i.e., higher temperature, than originally absorbed by the evaporator). The high-pressure superheated vapor discharged by the compressor, enters the condenser, and releases heat to form a high-pressure subcooled liquid. In the present invention, it was found possible to convert 40° C. hot water to get 80° C. hot water at 90° C. dew point at a pressure not exceeding 27 barg (bar gauge).

After leaving the condenser, the high-pressure liquid flows through the expansion element to form a low-pressure liquid due to the pressure drop that takes place at the element. During expansion, some vapor devolves from the liquid taking heat with it causing the temperature of the liquid to drop. Preferably, the enthalpy of the blend is substantially invariable during expansion. If desired or necessary, the high-pressure liquid can be cooled in a subcooler prior to conveyance to the expansion element to reduce the degree of flashing exhibited by the blend upon sudden expansion. After expansion at the expansion element, the low-pressure liquid (and vapor) enters the evaporator and is cycled again.

The heating/cooling apparatus of the present invention can take the form of any conventional vapor compression heating or cooling device or machine known in the art. Examples include heat pump systems, refrigerators, freezers, and chillers. The present invention is particularly useful in heat pump systems. A preferred chiller is a centrifugal chiller.

The following are examples of the present invention and are not to be construed as limiting. Unless otherwise indicated, all percentages and parts are by weight.

### EXAMPLES

Examples of working fluids of the present invention were prepared and their operating characteristics evaluated in a heat pump system. Their operating characteristics were compared to reference working fluids R114, R124 and 1-Chloro-1,1-difluoroethane (R142b).

The test equipment was a heat pump system. The system had a compressor, condenser, expansion element, and evaporator in series. The tests were carried out at the following conditions: 30° C. dew point temperature in the evaporator, 90° C. dew point temperature in the condenser, 10° C. superheating, 5° C. subcooling, 59.3% isentropic efficiency, an ideal heating coefficient of performance (COP) in the range of 2.8 to 3, and a 110° C. discharge temperature at the compressor. Data was collected using Refprop 7.1 software of the National Institute of Standards and Technology (NIST) at the specified conditions.

R245fa, R236ea, and R134a are the components in the working fluids of the examples. The components were physically admixed at ambient temperature at their designated mass percentages. Physical properties of the components are set forth below in Table 1.

TABLE 1
(Physical Data of the Components)

| | | Critical | Critical | | Boiling | | | |
|---|---|---|---|---|---|---|---|---|
| | Chemical | Temperature | Pressure | Molar | Point | | | |
| Component | Description | (° C.) | (bara) | Mass | (° C.) | ODP | GWP | Flammability |
| R134a | 1,1,1,2- | 101.06 | 40.593 | 102.03 | −26.074 | 0 | 1300 | N |
| | Tetrafluoro- | | | | | | | |
| | ethane | | | | | | | |
| R236ea | 1,1,1,2,3,3- | 139.29 | 35.02 | 152.04 | 6.19 | 0 | 1200 | N |
| | Hexafluoro- | | | | | | | |
| | propane | | | | | | | |
| R245fa | 1,1,1,3,3- | 154.05 | 36.4 | 134.05 | 14.9 | 0 | 950 | N |
| | Pentafluoro- | | | | | | | |
| | propane | | | | | | | |

bara—bar (absolute)
ODP—ozone depletion potential
GWP—global warming potential
N—nonflammable

Results are set forth in Tables 2 to 6. Examples are listed in 10% mass (weight) increments. Reference components R114, R124 and R142b were selected for performance comparison due to their relative large volumetric capacity and their non-ODP properties.

TABLE 2
(Comparative Examples 1 to 3 and Examples 1 to 5)

| Mixture Components | R114* | R124* | R142b* | Ex. 1 | Ex. 2 | Ex. 3 | Ex. 4 | Ex. 5 |
|---|---|---|---|---|---|---|---|---|
| Input | | | | | | | | |
| R245fa (mass %) | | | | 10% | 20% | 30% | 40% | 50% |
| R236ea (mass %) | | | | 80% | 70% | 60% | 50% | 40% |
| R134a (mass %) | | | | 10% | 10% | 10% | 10% | 10% |
| R245fa (mole %) | | | | 10.68% | 21.08% | 31.24% | 41.14% | 50.81% |
| R236ea (mole %) | | | | 75.30% | 65.06% | 55.08% | 45.34% | 35.84% |
| R134a (mole %) | | | | 14.03% | 13.85% | 13.68% | 13.51% | 13.35% |
| Conditions | | | | | | | | |
| Super Heat °K | 5 | 5 | 5 | 5 | 5 | 5 | 5 | 5 |
| Subcool °K | 5 | 5 | 5 | 5 | 5 | 5 | 5 | 5 |
| EVAP Dew Point temp ° C. | 30 | 30 | 30 | 30 | 30 | 30 | 30 | 30 |
| COND Dew Point temp ° C. | 90 | 90 | 90 | 90 | 90 | 90 | 90 | 90 |
| Isentropic efficiency | 0.593 | 0.593 | 0.593 | 0.593 | 0.593 | 0.593 | 0.593 | 0.593 |
| Volume Flow (M\hat{ }3/H) | 23.18 | 23.18 | 23.18 | 23.18 | 23.18 | 23.18 | 23.18 | 23.18 |
| Output | | | | | | | | |
| Cooling Capacity (KW) | 8.35 | 13.73 | 13.78 | 9.82 | 9.72 | 9.60 | 9.44 | 9.27 |
| Heating Capacity (KW) | 12.39 | 20.64 | 20.08 | 14.51 | 14.34 | 14.11 | 13.85 | 13.55 |
| Power Input (KW) | 4.04 | 6.91 | 6.30 | 4.69 | 4.62 | 4.52 | 4.40 | 4.28 |
| Discharge Temp ° C. | 96.17 | 105.46 | 113.47 | 98.84 | 99.49 | 100.17 | 100.90 | 101.64 |
| Cooling Capacity (KJ/Kg) | 71.88 | 79.95 | 124.92 | 93.03 | 95.82 | 98.95 | 102.37 | 106.04 |
| Heating Capacity (KJ/Kg) | 106.66 | 120.19 | 182.00 | 137.51 | 141.30 | 145.53 | 150.12 | 155.00 |
| Power Input (KJ/Kg) | 34.78 | 40.24 | 57.08 | 44.48 | 45.48 | 46.58 | 47.75 | 48.97 |
| COP for Cooling | 2.07 | 1.99 | 2.19 | 2.09 | 2.11 | 2.12 | 2.14 | 2.17 |
| COP for Heating | 3.07 | 2.99 | 3.19 | 3.09 | 3.11 | 3.12 | 3.14 | 3.17 |
| Volume Capacity (KJ/M³) | 1296.40 | 2132.12 | 2140.47 | 1524.88 | 1510.07 | 1490.30 | 1466.52 | 1439.91 |
| Pressure Ratio | 4.60 | 4.37 | 4.35 | 5.18 | 5.22 | 5.26 | 5.32 | 5.38 |
| Internal Volume Ratio | 4.59 | 4.33 | 3.96 | 5.15 | 5.15 | 5.16 | 5.17 | 5.19 |
| BP ° C. @ 1 bar | 3.25 | −12.28 | −9.43 | 3.41 | 4.13 | 5.03 | 6.04 | 7.13 |
| COND Pressure bar | 11.56 | 19.46 | 17.09 | 14.00 | 13.76 | 13.47 | 13.14 | 12.79 |
| COND Temp Glide | 0.00 | 0.00 | 0.00 | 4.42 | 4.54 | 4.77 | 5.08 | 5.44 |
| EVAP Temp Glide | 0.00 | 0.00 | 0.00 | 2.14 | 2.26 | 2.44 | 2.65 | 2.87 |
| ODP | 1.00 | 0.02 | 0.07 | 0.00 | 0.00 | 0.00 | 0.00 | 0.00 |
| GWP | 9800.00 | 620.00 | 2400.00 | 1187.34 | 1161.14 | 1135.59 | 1110.66 | 1086.33 |

*not an example of the present invention
M\hat{ }3/H—cubic meters per hour
KW—kilowatts
KJ—kilojoules
Kg—kilograms
M³—cubic meters
COND—condenser
EVAP—evaporator
°K—degrees Kelvin
BP—boiling point
temp—temperature
temp glide—glide temperature
COP—coefficient of performance

TABLE 3
(Examples 6 to 13)

| Mixture Components | Ex. 6 | Ex. 7 | Ex. 8 | Ex. 9 | Ex. 10 | Ex. 11 | Ex. 12 | Ex. 13 |
|---|---|---|---|---|---|---|---|---|
| Input | | | | | | | | |
| R245fa (mass %) | 60% | 70% | 80% | 10% | 20% | 30% | 40% | 50% |
| R236ea (mass %) | 30% | 20% | 10% | 70% | 60% | 50% | 40% | 30% |
| R134a (mass %) | 10% | 10% | 10% | 20% | 20% | 20% | 20% | 20% |
| R245fa (mole %) | 60.25% | 69.46% | 78.47% | 10.20% | 20.17% | 29.89% | 39.39% | 48.67% |
| R236ea (mole %) | 26.56% | 17.50% | 8.65% | 62.98% | 53.34% | 43.93% | 34.73% | 25.75% |
| R134a (mole %) | 13.19% | 13.04% | 12.89% | 26.81% | 26.49% | 26.18% | 25.88% | 25.58% |
| Conditions | | | | | | | | |
| Super Heat °K | 5 | 5 | 5 | 5 | 5 | 5 | 5 | 5 |
| Subcool °K | 5 | 5 | 5 | 5 | 5 | 5 | 5 | 5 |
| EVAP Dew Point Temp ° C. | 30 | 30 | 30 | 30 | 30 | 30 | 30 | 30 |
| COND Dew Point Temp ° C. | 90 | 90 | 90 | 90 | 90 | 90 | 90 | 90 |
| Isentropic efficiency | 0.593 | 0.593 | 0.593 | 0.593 | 0.593 | 0.593 | 0.593 | 0.593 |
| Volume Flow (M\hat{ }3/H) | 23.18 | 23.18 | 23.18 | 23.18 | 23.18 | 23.18 | 23.18 | 23.18 |
| Output | | | | | | | | |
| Cooling Capacity KW | 9.09 | 8.90 | 8.71 | 10.99 | 10.86 | 10.68 | 10.48 | 10.27 |
| Heating Capacity KW | 13.24 | 12.92 | 12.60 | 16.26 | 16.01 | 15.71 | 15.37 | 14.99 |
| Power Input KW | 4.15 | 4.02 | 3.89 | 5.27 | 5.16 | 5.03 | 4.88 | 4.73 |
| Discharge Temp ° C. | 102.38 | 103.11 | 103.82 | 101.55 | 102.21 | 102.93 | 103.69 | 104.47 |
| Cooling Capacity (KJ/Kg) | 109.88 | 113.84 | 117.85 | 96.56 | 99.67 | 103.18 | 107.03 | 111.16 |
| Heating Capacity (KJ/Kg) | 160.08 | 165.27 | 170.48 | 142.81 | 147.01 | 151.72 | 156.85 | 162.31 |
| Power Input (KJ/Kg) | 50.20 | 51.43 | 52.64 | 46.25 | 47.34 | 48.54 | 49.83 | 51.16 |
| COP for Cooling | 2.19 | 2.21 | 2.24 | 2.09 | 2.11 | 2.13 | 2.15 | 2.17 |
| COP for Heating | 3.19 | 3.21 | 3.24 | 3.09 | 3.11 | 3.13 | 3.15 | 3.17 |
| Volume Capacity (KJ/M3) | 1411.60 | 1382.46 | 1353.09 | 1707.40 | 1686.28 | 1659.44 | 1628.34 | 1594.64 |
| Press. Ratio | 5.44 | 5.50 | 5.55 | 5.14 | 5.19 | 5.25 | 5.31 | 5.38 |
| Internal Volume Ratio | 5.20 | 5.21 | 5.22 | 5.08 | 5.09 | 5.10 | 5.12 | 5.14 |
| BP Temp ° C. @ 1 bar | 8.26 | 9.40 | 10.53 | 0.50 | 1.38 | 2.43 | 3.60 | 4.84 |
| COND Pressure bar | 12.41 | 12.03 | 11.64 | 15.62 | 15.30 | 14.93 | 14.51 | 14.06 |
| COND Temp Glide | 5.82 | 6.18 | 6.49 | 6.41 | 6.66 | 7.06 | 7.57 | 8.16 |
| EVAP Temp Glide | 3.05 | 3.15 | 3.17 | 3.88 | 4.08 | 4.38 | 4.74 | 5.11 |
| ODP | 0.00 | 0.00 | 0.00 | 0.00 | 0.00 | 0.00 | 0.00 | 0.00 |
| GWP | 1062.57 | 1039.38 | 1016.72 | 1201.30 | 1176.08 | 1151.45 | 1127.40 | 1103.90 |

TABLE 4
(Examples 14 to 21)

| Mixture Components | Ex. 14 | Ex. 15 | Ex. 16 | Ex. 17 | Ex. 18 | Ex. 19 | Ex. 20 | Ex. 21 |
|---|---|---|---|---|---|---|---|---|
| Input | | | | | | | | |
| R245fa (mass %) | 60% | 70% | 10% | 20% | 30% | 40% | 50% | 60% |
| R236ea (mass %) | 20% | 10% | 60% | 50% | 40% | 30% | 20% | 10% |
| R134a (mass %) | 20% | 20% | 30% | 30% | 30% | 30% | 30% | 30% |
| R245fa (mole %) | 57.74% | 66.61% | 9.77% | 19.32% | 28.66% | 37.78% | 46.71% | 55.44% |
| R236ea (mole %) | 16.97% | 8.39% | 51.70% | 42.59% | 33.69% | 24.98% | 16.47% | 8.15% |
| R134a (mole %) | 25.29% | 25.00% | 38.52% | 38.08% | 37.65% | 37.23% | 36.82% | 36.42% |
| Conditions | | | | | | | | |
| Super Heat °K | 5 | 5 | 5 | 5 | 5 | 5 | 5 | 5 |
| Subcool °K | 5 | 5 | 5 | 5 | 5 | 5 | 5 | 5 |
| EVAP Dew Point Temp ° C. | 30 | 30 | 30 | 30 | 30 | 30 | 30 | 30 |
| COND Dew Point Temp ° C. | 90 | 90 | 90 | 90 | 90 | 90 | 90 | 90 |
| Isentropic efficiency | 0.593 | 0.593 | 0.593 | 0.593 | 0.593 | 0.593 | 0.593 | 0.593 |
| Volume Flow (M\hat{ }3/H) | 23.18 | 23.18 | 23.18 | 23.18 | 23.18 | 23.18 | 23.18 | 23.18 |
| Output | | | | | | | | |
| Cooling Capacity KW | 10.04 | 9.82 | 12.11 | 11.93 | 11.70 | 11.45 | 11.18 | 10.91 |
| Heating Capacity KW | 14.61 | 14.22 | 17.99 | 17.67 | 17.28 | 16.84 | 16.38 | 15.91 |
| Power Input KW | 4.57 | 4.40 | 5.89 | 5.74 | 5.58 | 5.39 | 5.20 | 5.00 |
| Discharge Temp ° C. | 105.24 | 105.99 | 104.11 | 104.80 | 105.58 | 106.40 | 107.23 | 108.05 |
| Cooling Capacity | 115.49 | 119.98 | 98.23 | 101.71 | 105.62 | 109.89 | 114.47 | 119.26 |
| (KJ/Kg) | | | | | | | | |
| Heating Capacity | 167.99 | 173.79 | 146.01 | 150.69 | 155.94 | 161.65 | 167.69 | 173.95 |
| (KJ/Kg) | | | | | | | | |
| Power Input (KJ/Kg) | 52.49 | 53.82 | 47.78 | 48.98 | 50.32 | 51.75 | 53.22 | 54.69 |
| COP for Cooling | 2.20 | 2.23 | 2.06 | 2.08 | 2.10 | 2.12 | 2.15 | 2.18 |
| COP for Heating | 3.20 | 3.23 | 3.06 | 3.08 | 3.10 | 3.12 | 3.15 | 3.18 |
| Volume Capacity | 1559.84 | 1525.01 | 1880.12 | 1852.16 | 1817.43 | 1778.11 | 1736.50 | 1694.46 |
| (KJ/M3) | | | | | | | | |
| Press. Ratio | 5.45 | 5.51 | 5.09 | 5.15 | 5.22 | 5.29 | 5.37 | 5.45 |
| Internal Volume Ratio | 5.16 | 5.17 | 5.00 | 5.01 | 5.04 | 5.06 | 5.09 | 5.11 |
| BP Temp ° C. @ 1 bar | 6.10 | 7.36 | −2.42 | −1.37 | −0.13 | 1.22 | 2.62 | 4.01 |
| COND Pressure bar | 13.60 | 13.14 | 17.38 | 16.97 | 16.49 | 15.97 | 15.43 | 14.87 |
| COND Temp Glide | 8.79 | 9.44 | 6.97 | 7.38 | 7.95 | 8.66 | 9.46 | 10.33 |
| EVAP Temp Glide | 5.45 | 5.71 | 5.00 | 5.34 | 5.80 | 6.35 | 6.92 | 7.45 |
| ODP | 0.00 | 0.00 | 0.00 | 0.00 | 0.00 | 0.00 | 0.00 | 0.00 |
| GWP | 1080.93 | 1058.48 | 1214.09 | 1189.77 | 1166.01 | 1142.77 | 1120.05 | 1097.83 |

TABLE 5
(Examples 22 to 29)

| Mixture Components | Ex. 22 | Ex. 23 | Ex. 25 | Ex. 26 | Ex. 27 | Ex. 28 | Ex. 29 |
|---|---|---|---|---|---|---|---|
| Input | | | | | | | |
| R245fa (mass %) | 10% | 20% | 40% | 50% | 10% | 20% | 30% |
| R236ea (mass %) | 50% | 40% | 20% | 10% | 40% | 30% | 20% |
| R134a (mass %) | 40% | 40% | 40% | 40% | 50% | 50% | 50% |
| R245fa (mole %) | 9.38% | 18.55% | 36.30% | 44.90% | 9.01% | 17.83% | 26.47% |
| R236ea (mole %) | 41.34% | 32.71% | 16.00% | 7.92% | 31.78% | 23.59% | 15.56% |
| R134a (mole %) | 49.28% | 48.74% | 47.69% | 47.19% | 59.20% | 58.58% | 57.97% |
| Conditions | | | | | | | |
| Super Heat °K | 5 | 5 | 5 | 5 | 5 | 5 | 5 |
| Subcool °K | 5 | 5 | 5 | 5 | 5 | 5 | 5 |
| EVAP Dew Point Temp ° C. | 30 | 30 | 30 | 30 | 30 | 30 | 30 |
| COND Dew Point Temp ° C. | 90 | 90 | 90 | 90 | 90 | 90 | 90 |
| Isentropic efficiency | 0.593 | 0.593 | 0.593 | 0.593 | 0.593 | 0.593 | 0.593 |
| Volume Flow (M\hat{ }3/H) | 23.18 | 23.18 | 23.18 | 23.18 | 23.18 | 23.18 | 23.18 |
| Output | | | | | | | |
| Cooling Capacity KW | 13.21 | 12.98 | 12.38 | 12.06 | 14.34 | 14.04 | 13.69 |
| Heating Capacity KW | 19.77 | 19.35 | 18.32 | 17.76 | 21.62 | 21.09 | 20.48 |
| Power Input KW | 6.56 | 6.37 | 5.94 | 5.70 | 7.28 | 7.05 | 6.79 |
| Discharge Temp ° C. | 106.48 | 107.24 | 109.01 | 109.92 | 108.63 | 109.50 | 110.48 |
| Cooling Capacity | 98.75 | 102.61 | 111.63 | 116.63 | 98.54 | 102.79 | 107.52 |
| (KJ/Kg) | | | | | | | |
| Heating Capacity | 147.78 | 153.01 | 165.15 | 171.80 | 148.57 | 154.39 | 160.85 |
| (KJ/Kg) | | | | | | | |
| Power Input (KJ/Kg) | 49.04 | 50.40 | 53.53 | 55.17 | 50.03 | 51.59 | 53.33 |
| COP for Cooling | 2.01 | 2.04 | 2.09 | 2.11 | 1.97 | 1.99 | 2.02 |
| COP for Heating | 3.01 | 3.04 | 3.09 | 3.11 | 2.97 | 2.99 | 3.02 |
| Volume Capacity (KJ/M3) | 2051.68 | 2015.62 | 1922.83 | 1872.21 | 2226.96 | 2181.15 | 2126.30 |
| Press. Ratio | 5.02 | 5.09 | 5.26 | 5.35 | 4.93 | 5.01 | 5.11 |
| Internal Volume Ratio | 4.91 | 4.93 | 5.00 | 5.04 | 4.80 | 4.84 | 4.88 |
| BP Temp ° C. @ 1 bar | −5.37 | −4.11 | −1.12 | 0.45 | −8.36 | −6.86 | −5.16 |
| COND Pressure bar | 19.26 | 18.74 | 17.52 | 16.87 | 21.24 | 20.60 | 19.90 |
| COND Temp Glide | 6.67 | 7.24 | 8.87 | 9.87 | 5.88 | 6.59 | 7.48 |
| EVAP Temp Glide | 5.50 | 6.01 | 7.44 | 8.23 | 5.46 | 6.17 | 7.06 |
| ODP | 0.00 | 0.00 | 0.00 | 0.00 | 0.00 | 0.00 | 0.00 |
| GWP | 1225.84 | 1202.37 | 1156.94 | 1134.95 | 1236.67 | 1213.99 | 1191.79 |

TABLE 6
(Examples 30 to 36)

| Mixture Components | Ex. 30 | Ex. 31 | Ex. 32 | Ex. 33 | Ex. 34 | Ex. 35 | Ex. 36 |
|---|---|---|---|---|---|---|---|
| Input | | | | | | | |
| R245fa (mass %) | 40% | 10% | 20% | 30% | 10% | 20% | 10% |
| R236ea (mass %) | 10% | 30% | 20% | 10% | 20% | 10% | 10% |
| R134a (mass %) | 50% | 60% | 60% | 60% | 70% | 70% | 80% |
| R245fa (mole %) | 34.93% | 8.67% | 17.17% | 25.50% | 8.36% | 16.56% | 8.07% |
| R236ea (mole %) | 7.70% | 22.94% | 15.14% | 7.49% | 14.74% | 7.30% | 7.11% |
| R134a (mole %) | 57.37% | 68.38% | 67.69% | 67.01% | 76.90% | 76.14% | 84.82% |
| Conditions | | | | | | | |
| Super Heat °K | 5 | 5 | 5 | 5 | 5 | 5 | 5 |
| Subcool °K | 5 | 5 | 5 | 5 | 5 | 5 | 5 |
| EVAP Dew Point Temp ° C. | 30 | 30 | 30 | 30 | 30 | 30 | 30 |
| COND Dew Point Temp ° C. | 90 | 90 | 90 | 90 | 90 | 90 | 90 |
| Isentropic efficiency | 0.593 | 0.593 | 0.593 | 0.593 | 0.593 | 0.593 | 0.593 |
| Volume Flow (M\hat{ }3/H) | 23.18 | 23.18 | 23.18 | 23.18 | 23.18 | 23.18 | 23.18 |
| Output | | | | | | | |
| Cooling Capacity KW | 13.31 | 15.51 | 15.14 | 14.70 | 16.71 | 16.26 | 17.93 |
| Heating Capacity KW | 19.82 | 23.55 | 22.90 | 22.16 | 25.54 | 24.76 | 27.57 |
| Power Input KW | 6.52 | 8.04 | 7.76 | 7.45 | 8.83 | 8.50 | 9.64 |
| Discharge Temp ° C. | 111.51 | 110.54 | 111.56 | 112.69 | 112.22 | 113.41 | 113.69 |
| Cooling Capacity (KJ/Kg) | 112.63 | 97.90 | 102.54 | 107.66 | 97.07 | 102.07 | 96.18 |
| Heating Capacity (KJ/Kg) | 167.79 | 148.68 | 155.12 | 162.23 | 148.38 | 155.44 | 147.88 |
| Power Input (KJ/Kg) | 55.16 | 50.78 | 52.58 | 54.57 | 51.31 | 53.38 | 51.70 |
| COP for Cooling | 2.04 | 1.93 | 1.95 | 1.97 | 1.89 | 1.91 | 1.86 |
| COP for Heating | 3.04 | 2.93 | 2.95 | 2.97 | 2.89 | 2.91 | 2.86 |
| Volume Capacity (KJ/M3) | 2066.60 | 2408.04 | 2350.85 | 2283.64 | 2594.61 | 2524.95 | 2784.53 |
| Press. Ratio | 5.22 | 4.82 | 4.92 | 5.04 | 4.70 | 4.82 | 4.58 |
| Internal Volume Ratio | 4.93 | 4.69 | 4.73 | 4.79 | 4.56 | 4.62 | 4.42 |
| BP Temp ° C. @ 1 bar | −3.40 | −11.40 | −9.60 | −7.63 | −14.47 | −12.32 | −17.53 |
| COND Pressure bar | 19.16 | 23.29 | 22.53 | 21.71 | 25.36 | 24.50 | 27.43 |
| COND Temp Glide | 8.53 | 4.83 | 5.66 | 6.68 | 3.71 | 4.62 | 2.60 |
| EVAP Temp Glide | 8.06 | 4.98 | 5.91 | 7.03 | 4.22 | 5.36 | 3.28 |
| ODP | 0.00 | 0.00 | 0.00 | 0.00 | 0.00 | 0.00 | 0.00 |
| GWP | 1170.04 | 1246.69 | 1224.75 | 1203.26 | 1255.99 | 1234.75 | 1264.64 |

The saturation pressure for the blend examples ranged from 19 to 27 bara (bar absolute) at a 90° C. dew point temperature. This is desirable because such blends can be employed as is in existing commercial compressors that are retrofitted for the use of R22, R134a and R404A, which also exhibit similar saturation pressures.

The blend examples also exhibited low boiling points at a pressure of one bar absolute. Low boiling points ensure that a heat pump system can hold a positive pressure not only during operation but also off-line and during shipping and storage. A positive system pressure prevents infiltration of air and moisture into the heat pump system and allows condensing pressure to be favorably controlled. For all the examples of this invention, the boiling point at one bar was lower than −5° C., so positive pressure could be maintained in heat pump system at high temperature conditions. If ambient temperatures fall below dew point temperature of the blend (e.g., during winter temperatures), then the heat pump should not be charged with the blend during shipping or storage.

Examples 27, 28, and 31 to 36 demonstrated effective performance and efficiency at high dew point condenser temperatures.

It should be understood that the foregoing description is only illustrative of the present invention. Various alternatives and modifications can be devised by those skilled in the art without departing from the invention. Accordingly, the present invention is intended to embrace all such alternatives, modifications and variances that fall within the scope of the appended claims.

What is claimed is:

1. A working fluid for a heating and cooling, comprising a blend of about 1% to about 98% by mass 1,1,1,3,3-pentafluoropane, about 1% to about 98% by mass 1,1,1,2,3,3-hexafluoropropane, and about 1% to about 98% by mass 1,1,1,2-tetrafluoroethane, wherein 1,1,1,3,3-pentafluoropane, 1,1,1,2,3,3-hexafluoropropane, and 1,1,1,2-tetrafluoroethane are about 90% or more by mass of the blend.

2. The working fluid of claim 1, comprising a blend of about 2% to about 40% by mass 1,1,1,3,3-pentafluoropane, about 2% to about 40% by mass 1,1,1,2,3,3-hexafluoropropane, and about 10% to about 85% by mass 1,1,1,2-tetrafluoroethane.

3. The working fluid of claim 1, comprising a blend of about 10% to about 40% by mass 1,1,1,3,3-pentafluoropane, about 10% to about 40% by mass 1,1,1,2,3,3-hexafluoropropane, and about 20% to about 70% by mass 1,1,1,2-tetrafluoroethane.

4. The working fluid of claim 1, wherein 1,1,1,3,3-pentafluoropane, 1,1,1,2,3,3-hexafluoropropane, and 1,1,1,2-tetrafluoroethane are about 95% or more by mass of the blend.

5. The working fluid of claim 1, wherein the blend further includes up to about 10% by mass of a hydrochlorofluorocarbon and/or a hydrofluorocarbon other than the foregoing.

6. The working fluid of claim 1, wherein the blend further includes up to about 5% by mass of a hydrochlorofluorocarbon and/or a hydrofluorocarbon other than the foregoing.

7. A heating/cooling apparatus, comprising a compressor, a condenser, an expansion element, and an evaporator in series in a cycle, wherein the apparatus further includes a working fluid of a blend of about 1% to about 98% by mass 1,1,1,3,3-pentafluoropane, about 1% to about 98% by mass 1,1,1,2,3,3-hexafluoropropane, and about 1% to about 98% by mass 1,1,1,2-tetrafluoroethane, wherein 1,1,1,3,3-pentafluoropane, 1,1,1,2,3,3-hexafluoropropane, and 1,1,1,2-tetrafluoroethane are about 90% or more by mass of the blend.

8. The apparatus of claim 7, wherein the working fluid is a blend of about 2% to about 40% by mass 1,1,1,3,3-pentafluoropane, about 2% to about 40% by mass 1,1,1,2,3,3-hexafluoropropane, and about 10% to about 85% by mass 1,1,1,2-tetrafluoroethane.

9. The apparatus of claim 7, wherein the working fluid is a blend of about 10% to about 40% by mass 1,1,1,3,3-pentafluoropane, about 10% to about 40% by mass 1,1,1,2,3,3-hexafluoropropane, and about 20% to about 70% by mass 1,1,1,2-tetrafluoroethane.

10. The apparatus of claim 7, wherein 1,1,1,3,3-pentafluoropane, 1,1,1,2,3,3-hexafluoropropane, and 1,1,1,2-tetrafluoroethane are about 90% or more by mass of the blend.

11. The apparatus of claim 7, wherein the blend further includes up to about 10% by mass of a hydrochlorofluorocarbon and/or a hydrofluorocarbon other than the foregoing.

12. The apparatus of claim 7 being a heat pump system.

13. The apparatus of claim 7 being a refrigerator.

14. The apparatus of claim 7 being a chiller.

15. A method for heating/cooling, comprising:
a) evaporating a working fluid in the form of a lower pressure liquid to form a lower pressure vapor;
b) compressing the lower pressure vapor to a higher pressure vapor;
c) condensing the higher pressure vapor to a higher pressure liquid;
d) expanding the higher pressure liquid to a lower pressure liquid; and
e) recycling the lower pressure liquid to step a),
wherein the working fluid includes a blend of about 1% to about 98% by mass 1,1,1,3,3-pentafluoropane, about 1% to about 98% by mass 1,1,1,2,3,3-hexafluoropropane, and about 1% to about 98% by mass 1,1,1,2-tetrafluoroethane.

16. The method of claim 16, wherein the working fluid is a blend of about 2% to about 40% by mass 1,1,1,3,3-pentafluoropane, about 2% to about 40% by mass 1,1,1,2,3,3-hexafluoropropane, and about 10% to about 85% by mass 1,1,1,2-tetrafluoroethane.

17. The method of claim 16, wherein the working fluid is a blend of about 10% to about 40% by mass 1,1,1,3,3-pentafluoropane, about 10% to about 40% by mass 1,1,1,2,3,3-hexafluoropropane, and about 20% to about 70% by mass 1,1,1,2-tetrafluoroethane.

18. The method of claim 16, wherein 1,1,1,3,3-pentafluoropane, 1,1,1,2,3,3-hexafluoropropane, and 1,1,1,2-tetrafluoroethane are about 90% or more by mass of the blend.

19. The method of claim 16, wherein the blend further includes up to about 10% by mass of a hydrochlorofluorocarbon and/or a hydrofluorocarbon other than the foregoing.

20. A method of cooling, comprising evaporating a blend of about 1% to about 98% by mass 1,1,1,3,3-pentafluoropane, about 1% to about 98% by mass 1,1,1,2,3,3-hexafluoropropane, and about 1% to about 98% by mass 1,1,1,2-tetrafluoroethane, wherein 1,1,1,3,3-pentafluoropane, 1,1,1,2,3,3-hexafluoropropane, and 1,1,1,2-tetrafluoroethane are about 90% or more by mass of the blend.

21. A method of heating, comprising condensing a blend of about 1% to about 98% by mass 1,1,1,3,3-pentafluoropane, about 1% to about 98% by mass 1,1,1,2,3,3-hexafluoropropane, and about 1% to about 98% by mass 1,1,1,2-tetrafluoroethane, wherein 1,1,1,3,3-pentafluoropane, 1,1,1,2,3,3-hexafluoropropane, and 1,1,1,2-tetrafluoroethane are about 90% or more by mass of the blend.
